(19) United States
(12) Patent Application Publication
(10) Pub. No.: US 2018/0168800 A1
(43) Pub. Date: Jun. 21, 2018

(54) INTRAOCULAR LENS INJECTOR CONTROL SYSTEM

(71) Applicant: PHYSIOL, Angleur (BE)

(72) Inventors: Fabian KAEGI, Lausanne (CH); Alain WOODTLI, Saint-Aubin-Sauges (CH); Christophe PAGNOULLE, Verviers (BE); Dimitriya BOZUKOVA, Liege (BE)

(73) Assignee: PHYSIOL, Angleur (BE)

(21) Appl. No.: 15/128,402

(22) PCT Filed: Mar. 27, 2015
(86) PCT No.: PCT/EP2015/056737

§ 371 Date: Sep. 22, 2016

(30) Foreign Application Priority Data
Mar. 28, 2014 (BE) 2014/0217

(51) Int. Cl. A61F 2/16

(52) U.S. Cl. CPC A61F 2/1672; A61M 5/14526; A61F 2/1678

(57) ABSTRACT

A control device for an intraocular lens injector characterized in that the device comprises a support arranged to accommodate a lens injector which may be connected to a cartridge optionally pre-loaded with a lens, said support comprising an actuator connected to a fluid transmission system, said actuator being arranged, as a result of the pressure force, to move between a first rest position and at least one second position.

The present invention relates to a control device for controlling an intraocular lens injector.

Intraocular lenses are intended to replace cloudy crystalline (natural) lenses in cataract patients, during a surgical operation. Cataract surgery is probably the most common surgical operation performed on humans, with a significant improvement in vision in 98% of patients.

Cataract surgery techniques have made enormous progress over the past few years, particularly due to the reduction in size of the incision through which the synthetic lens is implanted in the eye. New flexible materials (silicones, flexible acrylics and hydrogels) enable lenses to be implanted through smaller incisions than those made for the first rigid polymethyl methacrylate (PMMA) implants. A flexible lens can in effect be implanted in a folded or rolled conformation, before recovering its initial, definitive shape once placed in the eye, due to its special viscoelastic properties (shape memory). Small, suture-free incisions present the advantage of not creating corneal deformation, therefore preventing astigmatism. Restoration of vision is faster and the refractive outcome is more stable.

With the advent of flexible lenses, the problem of positioning these implants arose. In fact, to benefit from a reduced incision, the lens has to be folded and introduced in rolled form into the eye.

It is in that context that the applicant of the present application developed an injection device comprising an injector and a cartridge in which the lens is pre-loaded. This device was also the subject of the patent application published under number BE1016692.

An intraocular lens injector is disclosed in document US2009/0292293 A1. This injector includes a loading chamber, a distribution tube and a plunger rod to push the lens into the distribution tube.

The injection device injector may be, in particular, a syringe-type device mainly constituted of a hollow body (for example in ABS), a return spring and a plunger, which may be in polycarbonate, able to penetrate into the cartridge, and having on one of its ends, a sleeve arranged to cooperate and reversibly link with the intraocular lens. This injector, advantageously packaged separately from the cartridge, was designed at the time of implantation, to be, connected in a single action to the cartridge pre-loaded with the lens in a predictable and secure way.

The cartridge comprises:
A) a tapered cartridge body equipped with a loading area where the intraocular lens is loaded and in which it adopts an extended rest configuration (that is to say, unconstrained);
B) at a first end of the tapered cartridge body, an entrance through which the plunger can exert a pushing action on the intraocular lens placed in the loading area,
C) at a second end of the tapered cartridge body, an exit through which the intraocular lens can exit the cartridge;
D) a rolling area situated downstream from the loading area, between the loading area and the exit, in which the lens adopts a pre-folded configuration; and
E) an injection area defined between the exit and the rolling area of the cartridge.

In addition, the cartridge body comprises an inner cross-sectional shape that reduces from the entrance of the cartridge to the exit of the cartridge.

This injector is also intended to inject both hydrophilic and hydrophobic type lenses.

In practice, once the cartridge (pre-loaded with the lens in a loading area of this cartridge) is connected to the injector, the practitioner proceeds as follows:
i) Pre-folding step: The practitioner first applies a first pressure with the thumb on the injector plunger to displace it from a first rest position to a second lens rolling position. In fact, the lens is taken, through the movement of the plunger, from the cartridge loading area, where it is in a non-deformed configuration, to a rolling area, in part downstream from the inner cross-sectional shape of the cartridge defining the loading area, in which the intraocular lens adopts a pre-folded conformation; and
ii) Injection step: The practitioner again pushes or continues to push on the plunger so that the lens is forced out of said cartridge to the intraocular implantation site. In this step, the lens is taken from the cartridge rolling area in a pre-folded configuration, taking on the shape of a roller, to the injection area. As the exit also comprises a duct having the shape of decreasing cross-section from the rolling area to the outside, the lens progressively folds on itself when the plunger pushes the lens to the exit: During displacement of the lens from the loading area towards the injection area and the cartridge exit, the lens progressively goes from a pre-folded configuration to a rolled configuration in which the diameter reduces along the profile of the internal tapered section of the cartridge.

In practice, steps i) and ii) are performed in a single movement by the practitioner who continuously activates the plunger.

However, the practitioner can optionally perform reversing movements of the plunger from the injector during the injection step when he notices that there is a risk of catching the haptics. Any haptics accidentally wedged between the inner wall of the cartridge and the end of the injector plunger can thus be released.

In addition, besides the flexible lens placement problem resolved by the invention disclosed in patent BE1016692, there is another problem inherent to the use of intraocular lens injectors, including the injector as described in patent BE1016692: When using the lens injector during the injection step, the practitioner's thumb activates, via pressure exerted on the injector plunger, expulsion of the lens previously loaded into the cartridge connected to an injector connection area to the implantation site (that is to say, the eye). In so doing, the practitioner can exert considerable force on the injector plunger to expel the implant into the eye, which is especially the case when the practitioner injects a hydrophobic lens, perceived to be more rigid than a hydrophilic lens. This force will be greater the smaller the size of the corneal incision through which the lens must pass, or the higher the volume and/or rigidity of the lens.

Frequently, the force, sometimes necessarily excessive, exerted on the injector plunger can be transmitted at least partially to the eye, either due to accidentally placing part of the injector on the cornea, or due to a tremor of the practitioner, leading to lesions, sometimes irreversible, on the treated eye.

It is in this context that many devices for controlling an intraocular lens injector have been implemented. In particular, the literature mentions a control device for controlling an intraocular lens injector, said device comprising:
a control module, and
a hermetic incompressible fluid transmission system, connecting said control module to said injector, said control module being arranged to activate an injector by applying pressure to said transmission system fluid so as to generate a pressure force intended to operate the injector.

A control device for an intraocular lens injector as described above is known from document US2011/0264102A1 which discloses an intraocular lens injection device comprising an intraocular lens injector directly connected by a piping system to a hydraulic pedal, the injector plunger consequently being arranged to be hydraulically controlled by the action of the control pedal which is arranged to receive an electrical, mechanical or electromechanical signal and to convert it into a hydraulic pulse intended to exert a pressure force transmitted by the vector fluid to the injector plunger.

Unfortunately, the device from the prior art as described in document US2011/0264102A1 comprises a major disadvantage: The prior art device, through its design, does not ensure good airtightness of the hydraulic transmission system when the junction between the injector and the piping system is made, for example, when the injector has to be replaced after a single use. This then results in the presence of air bubbles that form in the vector fluid of the hydraulic system that can compromise the sensitivity of the control device, since it is known that air is a compressible fluid.

Therefore, at every use, the practitioner or technician must be especially careful not to allow air to infiltrate into the hydraulic transmission system when the injector is attached. Very often, a purging step is necessary to remove the undesirable air present in the hydraulic circuit, which makes the surgery preparation step particularly tedious.

The object of the invention is to mitigate the disadvantages from the prior art by providing a control device guaranteeing hermeticity of the hydraulic system when the injector is replaced, so as to facilitate its use.

To resolve this problem, a device as described above is provided according to the invention, characterized in that the device comprises a support arranged to accommodate the injector, said support comprising an actuator arranged to be in contact with and to cooperate with the injector, said actuator being connected to the fluid transmission system and, as a result of said fluid pressure force, arranged to be put into movement between a first rest position and at least one second position.

In this way, the hydraulic control device according to the invention guarantees airtightness of the hydraulic transmission system because the practitioner does not need to disconnect the transmission system at any time to replace the injector, since the injector actuator means is not in contact with the hydraulic transmission system fluid in any way.

In this context, when the injector is replaced, for example after a single use, the practitioner only has to disconnect the used injector by dislodging it from its support and replacing it with a new injector.

In addition, another advantage of the device according to the invention resides in that it is suitable for all types of injectors because, either the shape of the support can be easily adapted to the shape of the injector intended to be housed therein, or the support can take a standard shape that is compatible with several injector body shapes. For this purpose, the support comprises for example, a standard indentation, or an indentation that is predefined for a particular injector profile, this indentation being arranged to accommodate an injector intended to be detachably housed therein.

In addition, the control device according to the invention presents an additional advantage in that, when the cartridge is loaded into the injector, under the pressure force acting on the injector plunger, the practitioner only has to guide the tool in an appropriate manner to the implantation site and, for example, just by pressing the pedal with his foot, independently manage the pressure to be exerted on the fluid to implant the lens with considerable precision.

Thus, this system enables the force exerted by the surgeon's hand to be transferred to the injector plunger, for example by the foot of the practitioner, to guide the injector in the corneal incision, this guidance thereby becoming more precise as it is better controlled by the practitioner, who focuses his concentration and effort only on guiding and placing the implant in an adjusted manner.

Typically, the risk of excessive and traumatic pressure on the cornea is reduced during the lens implantation, which constitutes an important clinical advantage for the patient. In addition, the injection system enables the practitioner to limit the size of the incision to be made, this incision being set by the practitioner's judgment.

Another advantage inherent to the hydraulic control system is that it enables the injector plunger to be reversed more easily, by simply releasing the pedal, while maintaining optimal guidance of the implantation tool, so that the lens haptics can be released when they are accidentally wedged between the wall of the injector cartridge and the end of the plunger.

Advantageously, said incompressible fluid is a liquid.

In a first particular embodiment, said actuator presents a contact surface arranged to enter into contact with a surface of the injector, said contact surface being defined by a predetermined contact area value, and in that, for a predetermined pressure value constantly applied to said fluid through said control module, said pressure force of said fluid is proportional to said area value of said contact surface.

In a second particular embodiment, said actuator presents a contact surface arranged to enter into contact with a surface of the injector, said contact surface being defined by a predetermined contact area value, and in that, for a predetermined area value of said contact surface, said pressure force of said fluid is proportional to said predetermined pressure value constantly applied to said fluid through said control module.

In a particularly advantageous embodiment, the device according to the invention is characterized in that said fluid transmission system comprises at least one pipe communicating, at a first end, with said control module and, at a second end, with said actuator of said support, such that said fluid may circulate between said control module and said actuator.

Optionally, said transmission system comprises a first sealed part connected to the actuator of said support and intended to be connected by a connection means to at least one second sealed part of said transmission system, said connection means being intended to unseal each part of the transmission system to form a fluid transmission system which is hermetic and through which the fluid from each part of the transmission system is intended to circulate, said second sealed part of said transmission system being connected to a control module.

Preferably, said actuator comprises a piston assembled in a cylinder comprising a first end communicating with said fluid transmission system, said piston being lengthened by an arm which extends outside of said cylinder through an opening formed on a second end of the cylinder, opposite to the first end, said arm comprising an end arranged to enter in contact with and to cooperate with a part of an injector.

Other forms of embodiment of the device according to the invention are indicated in the appended claims.

The present invention thus relates to a support arranged to accommodate an intraocular lens injector, said support comprising an actuator arranged to be in contact and cooperate with a part of an injector, said actuator being arranged to be connected through an incompressible fluid transmission system to a control module, said actuator being arranged to move, as a result of a fluid pressure force generated from pressure applied to said fluid by said control module, between a first rest position and at least one second position.

In particular, the support comprises an indentation arranged to accommodate an injector intended to be detachably housed therein.

Advantageously, said incompressible fluid is a liquid.

In a first particular embodiment, said actuator presents a contact surface arranged to enter into contact with a surface of the injector, said contact surface being defined by a predetermined contact area value, and in that, for a predetermined pressure value constantly applied to said fluid through said control module, said pressure force of said fluid is proportional to said area value of said contact surface.

In a second particular embodiment, said actuator presents a contact surface arranged to enter into contact with a surface of the injector, said contact surface being defined by a predetermined contact area value, and in that, for a predetermined area value of said contact surface, said pressure force of said fluid is proportional to said predetermined pressure value constantly applied to said fluid through said control module.

In a particularly advantageous embodiment, the support according to the invention is characterized in that said fluid transmission system comprises at least one pipe communicating, at a first end, with said control module and, at a second end, with said actuator of said support, such that said fluid may circulate between said control module and said actuator.

Optionally, said transmission system comprises a first sealed part connected to the actuator of said support and intended to be connected by a connection means to at least one second sealed part of said transmission system, said connection means being intended to unseal each part of the transmission system to form a fluid transmission system that is hermetic and through which the fluid from each part of the transmission system is intended to circulate, said second sealed part of said transmission system being connected to a control module. The first single-use part may advantageously be provided sterile, while the second part is reusable.

Preferably, said actuator comprises a piston assembled in a cylinder presenting a first end communicating with said fluid transmission system, said piston being lengthened by an arm that extends outside of said cylinder through an opening formed on a second end of the cylinder, opposite to the first end, said arm comprising an end arranged to enter in contact with and to cooperate with a part of an injector.

Other forms of embodiment of the support according to the invention are indicated in the appended claims.

The invention also relates to a first assembly comprising:
the device according to the invention, and
an intraocular lens injector.

The invention also relates to a second assembly comprising:
the support according to the invention, and
an intraocular lens injector.

In particular, this second assembly also comprises:
a control module, and
an incompressible fluid transmission system arranged to connect said control module to said support.

Preferentially, the first or the second assembly comprises a cartridge in which an intraocular lens is pre-loaded in an unconstrained (at rest) configuration, said cartridge being arranged to be assembled on a connection area of the injector. In a particular embodiment, the cartridge may be assembled on said connection area of the injector.

In particular, the injector is housed in the support.

Other forms of embodiment of the first and second assemblies according to the invention are indicated in the appended claims.

Another object of the invention is a method for operating the control device comprising:
1) a step in which said control module is activated so as to apply pressure on said fluid;
2) a step in which, as a result of the pressure applied by said control module, a pressure force is generated, this pressure force being intended to operate the injector.

This method is characterized in that said pressure force is exerted on the injector through the actuator of said support, said actuator, as a result of said fluid pressure force, being arranged to be moved between a first rest position and at least one second position.

Advantageously, this method comprises a step in which a cartridge is assembled on a connection area of the injector, said cartridge comprising an intraocular lens which is pre-loaded therein.

It is well understood that the lens may also be manually loaded by the operator according to the standard method known to the person skilled in the art.

Preferably, the method according to the invention is characterized by the following successive steps:
a first pre-folding step, in which said control module is activated so that the actuator is displaced from said first rest position to a second lens rolling position corresponding to a first state in which the lens is housed in pre-folded form in a rolling area defined in one part of the inner wall of said cartridge; and
A second injection step, in which said control module is activated so that the actuator is displaced from said second lens rolling position to a third injection position, said second step corresponding to a second state in which said lens is driven outside of said cartridge.

Optionally, the operating method is also characterized in that, during the second injection step, said control module is activated so that said actuator is displaced from said third injection position to said second lens rolling position.

Other forms of embodiment of the method according to the invention are indicated in the appended claims.

Other characteristics, details and advantages of the invention will emerge from the following non-limiting description, with reference to the appended drawings.

FIG. 1 is a schematic view illustrating the device according to the invention.

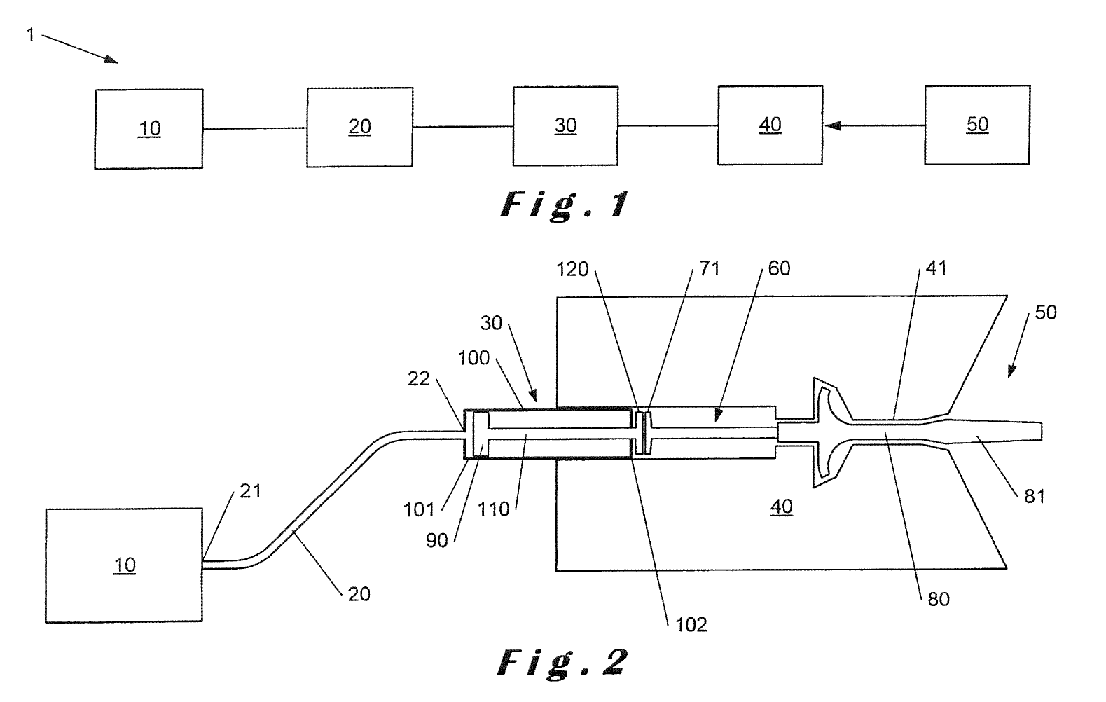

FIG. 2 is a schematic view of a first variation of the device according to the invention.

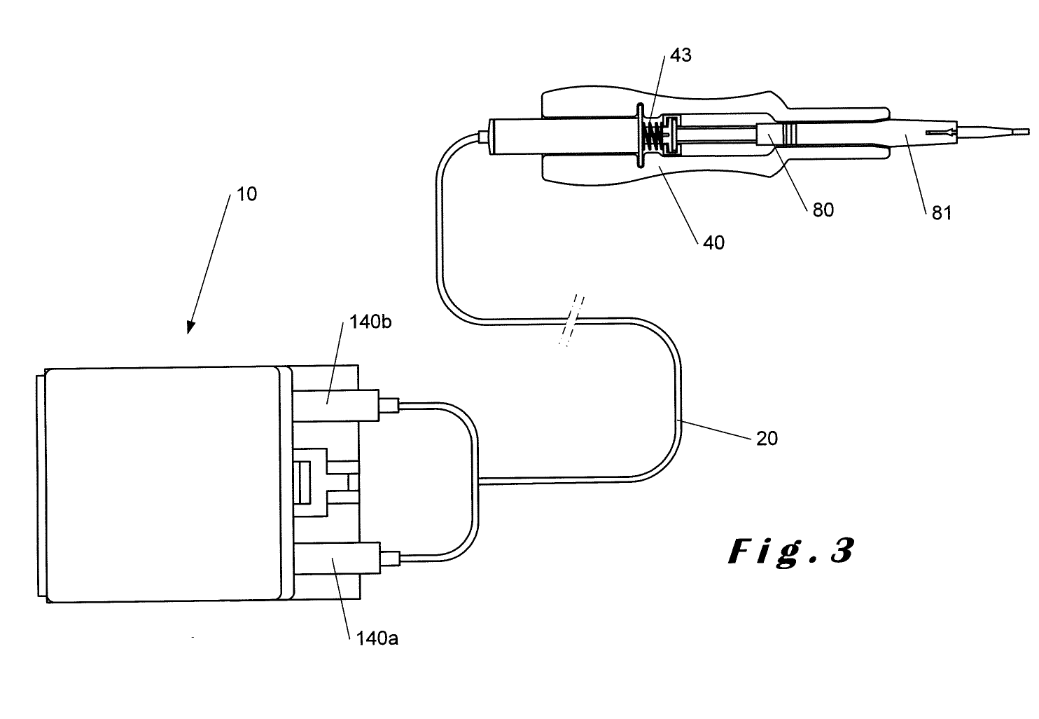

FIG. 3 is a top view of another variation of the device according to the invention.

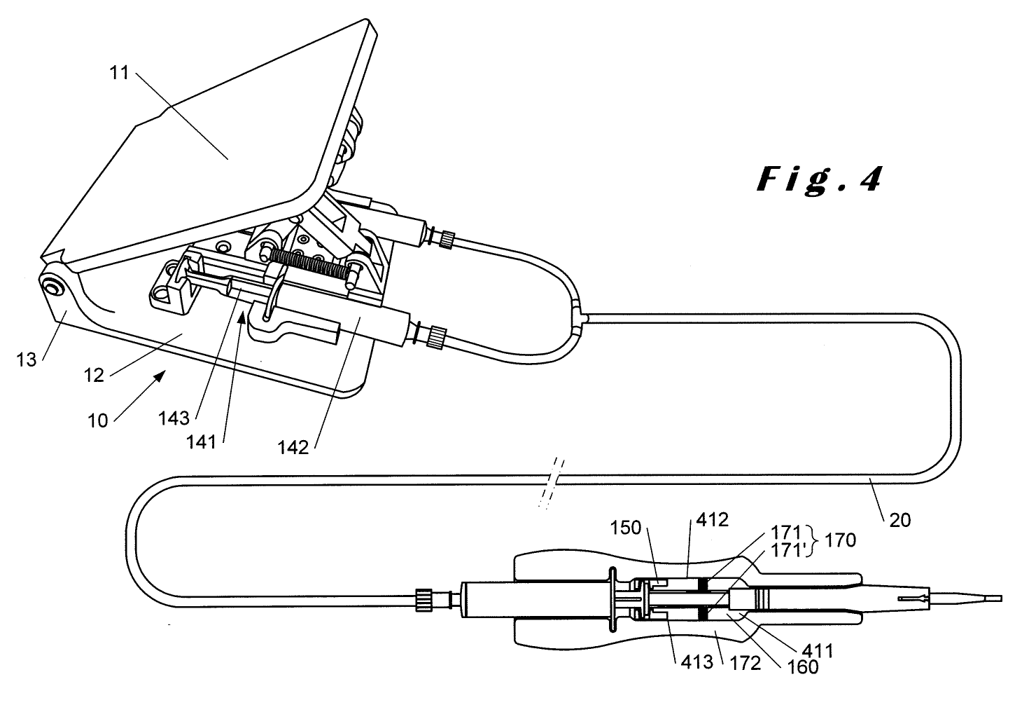

FIG. 4 is a perspective view of a first preferential mode of embodiment of the device according to the invention.

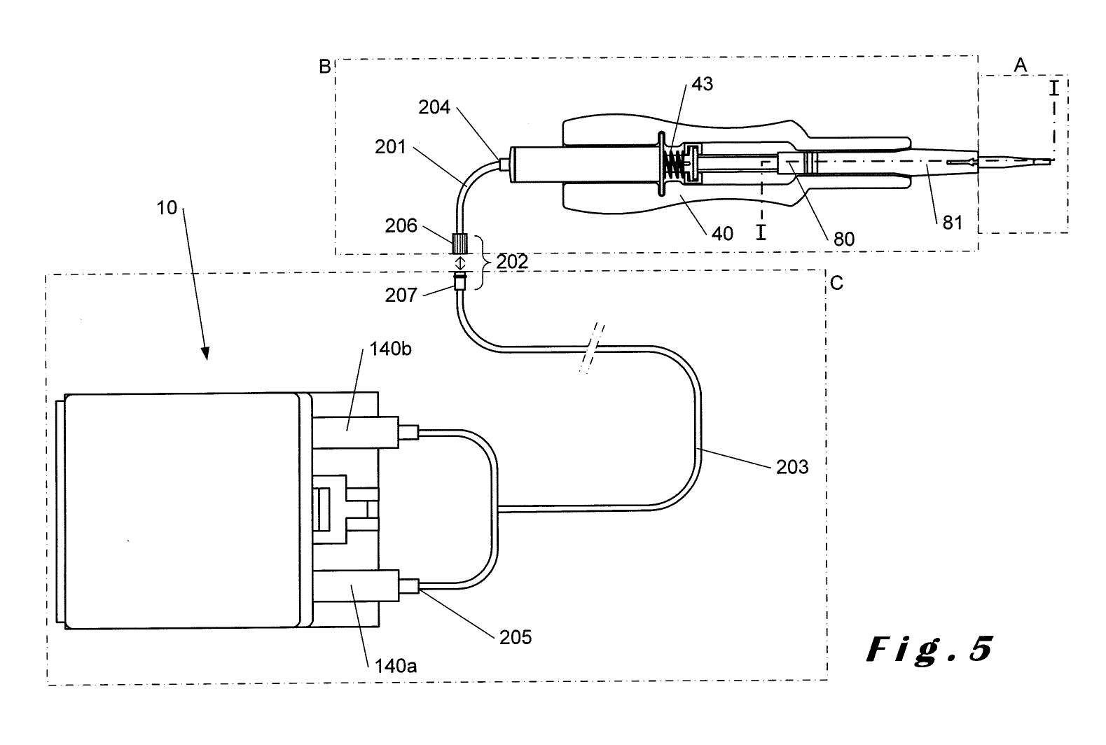

FIG. 5 is a top view of another mode of embodiment of the device according to the invention.

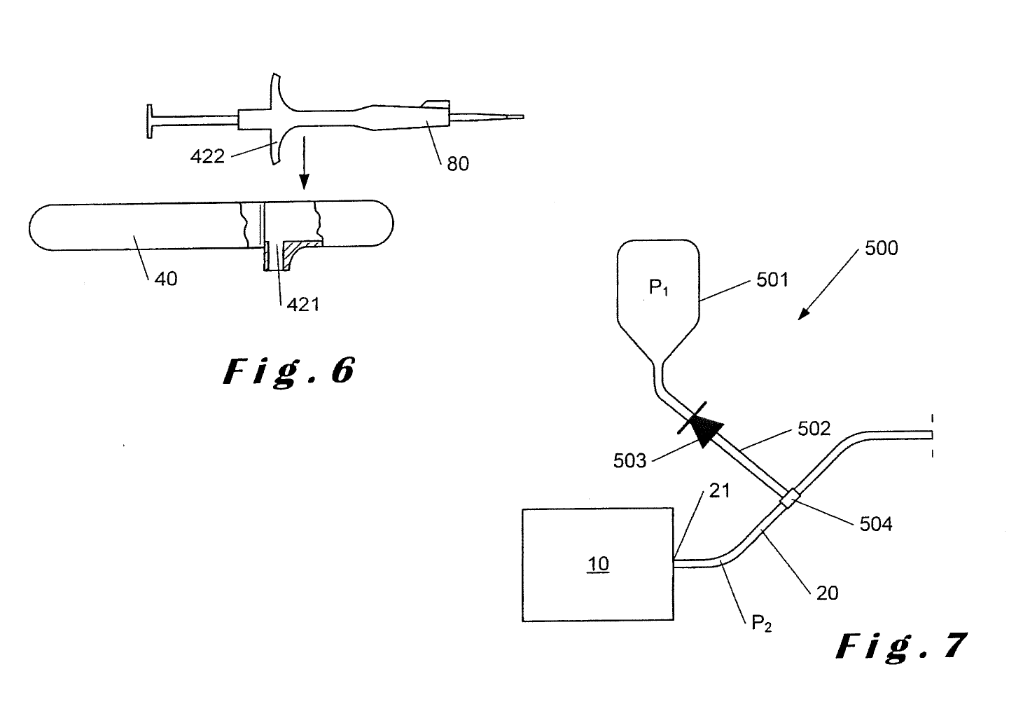

FIG. 6 is a longitudinal cross-sectional view (I-I) of the support from FIG. 5.

FIG. 7 is a schematic view of a particular mode of embodiment of the device according to the invention.

In the figures, identical or similar elements bear the same references.

FIG. 1 schematically illustrates control device 1 for an intraocular lens injector 50 according to the invention.

Device 1 comprises:
a control module 10, which may for example be a control pedal;
a support 40, arranged to accommodate an intraocular lens injector 50;
an actuator 30 of support 40 connected through a hermetic fluid transmission system 20 to control module 10, arranged to cooperate with a part of injector 50 which may be an actuator means, for example a mobile piston system housed in a cylinder.

Optionally, the transmission system comprises several parts arranged to be interconnected and to form thus a hermetic fluid hydraulic transmission system.

FIG. 2 is a schematic view of a variation of the control device 1 according to the invention in which the control module 10 is arranged to be connected to an actuator means (or mobile plunger) 60 of intraocular lens injector 50 through the fluid transmission system 20.

In FIG. 2, the fluid transmission system 20 comprises at least one pipe communicating, at a first end 21, with the control module 10 and, at a second end 22, with actuator 30 of support 40, so that the fluid can circulate between the control module 10 and the actuator 30 of support 40. The transmission system is preferably a piping system.

In particular, the fluid contained in transmission system 20 is an incompressible fluid, advantageously water or oil, the control module 10 then being a hydraulic pedal.

In FIG. 2, the mobile plunger 60 of the injector is assembled on a body 80 extended by a connection head 81 forming a connection area arranged to receive a cartridge in which a lens is pre-loaded and from which the lens is injected. In particular, plunger 60 is arranged to be moved from a first rest position to a second lens rolling position or from the second position to a third lens injection position.

The injector plunger extends outside of body 80 of the injector, opposite to the connection head 81, and comprises a first end 71.

In particular, actuator 30 of support 40 comprises a piston 90 assembled in cylinder 100 presenting a first end 101 communicating with the second end 22 of the piping system 20, so that the fluid can circulate through the piping, between the control module and piston 90 assembled in cylinder 100, and enter into contact with the cylinder 100.

As illustrated in FIG. 2, piston 90 is lengthened by an arm 110 that extends outside of cylinder 100 through an opening provided on the second cylinder end 102, opposite from the first cylinder end 101, and which comprises an end 120 arranged to be in contact with the first end 71 of injector plunger 60.

A contact surface, having a predetermined area value, is defined at end 120 of the arm of piston 90 of actuator 30 and is arranged to be in contact with a surface of the first end 71 of injector 50 plunger 60.

The device is characterized in that, for a predetermined pressure value constantly applied to the fluid through said control module, the pressure force of the fluid is proportional to the contact surface area value.

Alternatively, the device is also characterized in that, for a predetermined contact surface area value, said pressure force of said fluid is proportional to said predetermined pressure value constantly applied to said fluid through said control module.

In practice, the force required to effectively inject the intraocular lens is between 10 N and 50 N, depending on the nature of the lens. The magnitude of this pressure force will be higher the smaller the mean diameter of the distal opening of the cartridge through which the lens is injected for a smaller corneal incision, the higher the volume of the implant (high focal strength), and the more rigid the material constituting the lens.

Typically, for a hydrophilic type lens, known to be less rigid than a hydrophobic lens, a force of 10 N is sufficient, while for a hydrophobic lens, a force at least equal to 30 N will be required to inject the lens.

As an illustration, if the contact surface formed between the actuator and the actuator means is between 0.80 cm² and 1.50 cm², the pressure generated by the hydraulic pedal on the fluid to inject a hydrophilic lens must be between 6.67 Ncm² and 12.5 Ncm².

To inject a hydrophobic lens, the pressure must be at least three times higher, preferably between 33.3 Ncm² and 62.5 Ncm².

It should be noted in the context of the present invention that the hydraulic pedal may comprise one or more actuator means.

Therefore, in a preferential mode of embodiment as illustrated in FIGS. 3 and 4, the control module 10 is a hydraulic pedal arranged to accommodate at least one actuator means 140a. In the particular case as described in FIGS. 3 and 4, the hydraulic pedal includes two actuator means 140a, 140b, each comprising a piston 141 housed in a cylinder 142 and extended by an arm 143.

The hydraulic pedal comprises a plate 11 connected to a pedal lever 12 arranged to cooperate with the arm of each actuator means, and a base 13 connected by a connecting axis to the plate arranged to pivot with respect to the base on the connecting axis between, successively, a first pivot position and a second pivot position, and between said second (intermediate) pivot position and a third pivot position.

In a preferential mode of embodiment, the plate is arranged to pivot with respect to the base on the connecting axis between said first pivot position and said third pivot position.

In this context, the first pivot position of the pedal corresponds to the first rest position of injector plunger 60, the second (intermediate position) pivot position corresponds to the second position of injector plunger 60 (pre-folding step), while the third pivot position corresponds to the third position of injector plunger 60 (injection step).

The use of a hydraulic pedal as a control means is particularly advantageous given that it is well known that the pressure generated by the force of the foot applied to a predetermined surface is on average ten times higher than that generated by the thumb. In this context, the practitioner has no difficulty in generating a pressure force of between 10 N and 50 N by simply moving his foot, and which can then be perfectly measured.

Piston 90 of actuator 30 of support 40 is arranged to be moved in cylinder 100, from a first rest position, corresponding to a first state in which the injector plunger 60 is at rest, to a second position corresponding to a second state in which the injector plunger 60 is in a pre-folding position and a third position corresponding to a third state in which the injector plunger 60 is in an injection position.

The support also presents an indentation 41 arranged to accommodate the injector that is intended to detachably fit therein, the shape of the indentation 41 preferably being substantially complementary to that of the injector body.

Preferably, the indentation is a throat formed in the support in which the injector is inserted by embedding and/or sliding.

Alternatively, the indentation is adjustable so as to conform to the shapes of the injector body.

In another mode of embodiment, support 40 comprises a movable joining means 150, placed in the space defined by indentation 41, and arranged to connect the end 120 of arm 110 of piston 90 of actuator 30 to end 71 of injector plunger 60, the joining means 50 being arranged to displace in one direction corresponding to that of the displacement of the actuator piston arms and the injector.

Preferably, support 40 comprises a means 160 for guiding the movable joining means 150. This guiding means may be for example a guide rail housed in indentation 41 and presenting one direction corresponding to that of the displacement of the movable joining means 150 between the first rest position and the second pre-folding position and between the second pre-folding position and the third injection position, the movable joining means 150 being a base arranged to be assembled on the rail and to move along the rail.

Alternatively, indentation 41 comprises a throat in which the movable joining means 150 is housed. The throat comprises a bottom wall 411 connected to two lateral walls 412, 413 opposite each other that extend from bottom wall 411 in a direction substantially perpendicular to a plane tangential to bottom wall 411.

In this context, the throat obtains the means 160 for guiding the joining means 150.

The bottom wall and lateral walls thus form a throat with a U-shaped cross section and provides a frontal access opening opposite the bottom wall, giving access to the bottom wall and by which the injector body is brought to be housed in the throat.

Each lateral wall comprises an inner surface, oriented to said movable joining means 150. Each inner surface of each inner wall comprises a groove, the movable joining means provides a pair of tongues, each tongue being arranged to cooperate with one of the grooves and be housed therein. In this context, the tongues and grooves define the means for guiding the movable joining means.

Optionally, for reasons of security, support 40 is also equipped with a security system 170 comprising a pair of end stops 171, 171′. For example, each stop is a bead projecting from each inner surface of each lateral wall 412, 413 of indentation 41.

Each end stop 171 is also placed at a stop position 172 on each lateral wall 412, 413, this stop position corresponding to the third injection position of piston 71 of injector 50.

In addition, in a preferential mode of embodiment, each stop of the security system 170 is placed at a first end of each groove present on each lateral wall of the indentation, each first groove end being placed at the stop position.

In this way, piston 71 of injector 50, when it is displaced from the first rest position to the third injection position, cannot go beyond a threshold position fixed by the stop position, such that the risk of creating excessive pressure exerted on the control module 10 resulting in a sudden, uncontrolled displacement of the injector piston is reduced. Consequently, the risk of trauma to the cornea due to a too-sudden injection of the intraocular lens is de facto reduced.

Advantageously, the device is equipped with a retraction system arranged to bring the piston of actuator 30 of support 40 from the second position or from the third position to the first position or enabling reversing movements at any time of the injector piston by simply releasing the pedal to release the lens haptics which may accidentally be wedged between the inner wall of the cartridge and the end of the injector plunger.

In particular, this retraction system comprises at least one extension spring 43 connecting end 120 of arm 110 of piston 90 to cylinder 100 of actuator 30, so that when the piston 90 is in the second pre-folding position or in the third injection position, the spring is extended and exerts a retraction force on piston 90 from the third injection position to the first rest position.

Alternatively, the extension spring 43 is arranged to connect the movable joining means 150 to cylinder 100 of actuator 30.

In an alternative embodiment, the retraction system comprises at least one compression spring connecting plate 11 to base 13 of hydraulic control pedal 11, such that when the plate of the pedal is in the third pivot position, the spring is compressed and exerts a retraction force on the plate, the retraction force being oriented from the third injection position to the first rest position.

In this way, the pressure exerted on the injector piston is managed by the pressure exerted on the pedal, which presses on the piston of said at least one actuator means, thereby propelling the fluid through the transmission system, this fluid thereby exerting proportional pressure to the piston of the support connected to the injector piston. Conversely, when the pedal is released, the piston of said at least one actuator means releases, as a result of the retraction force, the fluid is transferred to the cavity formed in the body of the actuator means and no longer exerts pressure on the support piston.

In a particular mode of embodiment as illustrated in FIG. 5, actuator 30 of support 40 is connected to a first sealed part 201 of the fluid transmission system 20. This first part is intended to be connected by a connection means 202 to a second sealed part 203 of the fluid transmission system 20 that is connected to control module 10. The connection means 202 is intended to unseal each part of the transmission system 20 to form a fluid transmission system that is hermetic and through which the fluid from each part of the transmission system can circulate.

In particular, a first end 204 of this first part of transmission system 201 communicates with actuator 30 of support 40, while said at least one second part of fluid transmission system 203 comprises a first end 205 that communicates with control module 10. Each part 201, 203 of the transmission system also comprises a second sealed end 206, 207, the second sealed ends being arranged to be interconnected by connection means 202 intended to unseal each part of transmission system 20 to form a fluid transmission system that is hermetic and through which the fluid from each part of the transmission system is intended to circulate. For example, this connection means 202 is a valve with at least 2 ports, of the infusion port type, arranged to be connected to each second sealed end 206, 207. Preferably, each valve port comprises a male or female end piece arranged to be fitted in a sealed female or male end piece placed on the second end of each part of the transmission system.

Alternatively, actuator 30 of support 40 is connected to the first end 22 of transmission system 20, the second end 21 of the transmission system 20 being arranged to be connected to a first connection means present on control module 10. For example, the transmission system is a pipe, optionally comprising different interconnected parts connected by at least one connection means 202, providing a first end connected to cylinder 100 of support actuator 30, a second end of the pipe being arranged to be connected to an end piece of actuator means 140a, 140b of the control module assembled on the hydraulic pedal.

In addition, in an alternative mode of embodiment, control means 10 is connected to the first end 22 of transmission system 20, the second end 21 of the transmission system 20 being arranged to be connected by a second connection means to actuator 30 of support 40. For example, the transmission system is a pipe, optionally comprising different interconnected parts connected by at least one connection means 202, presenting a first end connected to an end piece of an actuator means 140a, 140b of the control module assembled on the hydraulic pedal, and a second end being arranged to be connected to cylinder 100 of actuator 30 of support 40.

Preferably, the support comprises a reversible injector fixation means. In the preferential mode of embodiment as illustrated in FIG. 6, this fixation means 42 comprises, first, an opening 421 present on the support, preferably traversing indentation 41 and, second, a projecting protuberance 422 present on body 80 of injector 50 arranged to traverse and be housed in opening 421 present on support 40.

In a preferential mode of embodiment of the present invention as illustrated in FIG. 7, control device 1 according to the invention comprises an anti-bubble module 500 intended to prevent the infiltration of ambient air into the hydraulic transmission system 20.

The air infiltration phenomenon is observed in particular with transmission means 20 made from a plastic material (such as PVC pipes) that tend to become porous and permeable with time. In particular, in conjunction with the evaporation of part of the vector fluid contained in the pipe(s) through the pipe walls, the infiltration of air through these same walls and the formation of air bubbles in the hydraulic transmission system is observed, this phenomenon having the consequence of disrupting the proper operation of the control device, the air bubbles acting as series springs that can in fact limit the sensitivity of the control device or, more dramatically, compromise its operation.

In this context, anti-bubble module 500 is intended to compensate for the loss of vector fluid that evaporates through the walls of transmission system 20. In the mode of embodiment as illustrated in FIG. 7, this anti-bubble module 500 comprises a pressurized reservoir 501 (for example, a tank) comprising a first vector fluid part, this reservoir being connected by a second transmission system 502 to transmission system 20 through a second connection means 504, so that the vector fluid can circulate between reservoir 501 and transmission system 20 of control device 1.

Anti-bubble module 500 also comprises a non-return valve 503 to control the direction of circulation of the vector fluid by enabling the passage of this fluid in a first direction defined from reservoir 501 to said transmission system 20, while blocking the circulation of fluid in a direction reverse to the first direction.

The presence of this non-return valve consequently prevents overpressure at the level of the reservoir when the control device is in operation.

In practice, the vector fluid contained in reservoir 501 comprises a first pressure (P₁) greater than a second pressure (P₂) of a second vector fluid part present in transmission system 20. Preferably, the pressure difference value ΔP=P₁−P₂ is at least equal to a second pressure difference value between the second pressure value (P₂) inside the transmission system 20 and a third pressure measured outside of the transmission system (atmospheric pressure).

In this way, when a first volume of a first vector fluid part evaporates through the walls of transmission system 20, it is instantly replaced by a second equivalent volume of a second vector fluid part contained in reservoir 501, thereby preventing the diffusion of ambient air from the outside to the inside of transmission system 20.

The present invention also applies to a first assembly comprising device 1 according to the invention and an intraocular lens injector 50, preferably connected to support 40.

The present invention also applies to a second assembly comprising support 40 according to the invention and an intraocular lens injector 50. Preferably, injector 50 is connected to support 40.

In particular, the second assembly comprises a control module 10, optionally an anti-bubble module 500, and an incompressible fluid transmission system 20 arranged to connect control module 20 to actuator 30 of support 40 to form control device 1.

Preferably, the transmission system comprises first and second sealed parts 201, 203. The first sealed part 201 is connected, at a first end 204, to actuator 30 of support 40. Preferably, the first sealed part is also connected to said anti-bubble module 500. The second sealed part 203 is connected, at a first end 205, to control module 10. The second ends 206, 207 of each part of the transmission system 201, 203 are arranged to be interconnected by connection means 202. This connection means is intended to unseal each part of transmission system 20 to form a fluid transmission system that is hermetic and through which the fluid from each part of the transmission system is intended to circulate.

Advantageously, the first or the second assembly comprises a cartridge in which an intraocular lens is pre-loaded in a loading area. This cartridge is arranged to be assembled on a connection area of the injector. Preferentially, the cartridge is assembled on the connection area of injector 50. The cartridge is assembled on the injector connection area by a connection means consisting of a fin which, when the cartridge is pushed into the injector connection area, is adjustably housed in a slot of the injector whose shape is complementary to that of the fin.

Preferably, in support 40 of device 1, at least the first sealed part 201 of incompressible fluid transmission system 20, and optionally injector 50 (sub-assembly B in FIG. 5) are intended for single use and are therefore contained in sterile packaging.

Alternatively, injector 50 is contained in separate sterile packaging and is therefore not assembled on support 40.

Cartridge A (optionally pre-loaded with the lens, FIG. 5), that was not previously assembled on the injector connection area, is packaged separately in sterile packaging.

In this context, sub-assembly C (FIG. 5) comprising control module 10 and the second sealed part 201 of the incompressible fluid transmission system 20 is defined as being a sub-assembly of the device that is reusable and therefore not sterile.

In particular, injector 50 and the cartridge are the subject of patent BE1016692.

In operation, control module 10 is activated so as to apply pressure to the fluid in order to generate pressure force creating fluid movement through transmission system 20, between control module 10 and the actuator means of injector (plunger) 60. The pressure force is exerted on actuator means 60 of injector 50 through actuator 30 of support 40, so that actuator means 60 is displaced between the first rest position and the pre-folding position of the lens from a cartridge previously assembled on a connection area of the injector, the lens being pre-loaded into the cartridge, and between the second pre-folding position and the third injection position of the lens.

In particular, the operation of the device according to the invention is defined by the following successive steps:
a first pre-folding step, in which said control module 10 is activated so that actuator 30 is displaced from said first rest position to a second lens pre-folding position corresponding to a first state in which the lens is housed in pre-folded form in a rolling area defined in one part of the inner section of said cartridge; the intraocular lens is in fact brought from the cartridge loading area where it is found in an unconstrained configuration to the rolling zone in which the lens is in a pre-folded configuration; and
a second infection step, in which said control module is activated so that the actuator is displaced from said second lens rolling position to a third injection position, said second step corresponding to a second state in which said lens is driven outside of said cartridge: During displacement of the lens from the loading area towards the injection area and the cartridge exit, the lens progressively goes from a pre-folded configuration to a rolled configuration in which the diameter reduces along the profile of the internal tapered section of the cartridge. Advantageously, prior to the step in which control module 10 is activated, the operation of the device according to the invention also involves a third additional step in which the cartridge is assembled on the injector connection area.

The operating method is also characterized in that, during the second injection step, control module 10 can be activated so that actuator means 60 of injector 50 is displaced from the third injection position to the second lens pre-folding position.

In practice, it may in fact be useful to release the hydraulic control pedal to enable the injector actuator means to operate a sufficient retraction so that the undesirable haptics that are accidentally captured between the inner wall of the cartridge and the actuator means can be released.

Preferably, the operation of the device according to the invention also comprises a step in which, when a difference value ΔP between a first pressure value P₁ of a first vector fluid part contained in the reservoir 501 of the anti-bubble module 500 and a second pressure value P₂ of a second vector fluid part contained in the transmission system 20 of the control device 1 is at least equal to a threshold value, the vector fluid contained in the reservoir 501 circulates in a first direction, from said reservoir 501 to said transmission system 20, so that the difference value ΔP is less than said threshold value.

In this way, when a first volume of a first vector fluid part evaporates through the walls of transmission system 20, it is instantly replaced by a second equivalent volume of a second vector fluid part contained in reservoir 501, thereby preventing the diffusion of ambient air from the outside to the inside of transmission system 20 and the formation of air bubbles in the transmission system.

Advantageously, pressure difference value ΔP=P₁−P₂ is at least equal to a second pressure difference value between the second pressure value P₂ inside the transmission system 20 and a third pressure measured outside the transmission system.

Advantageously, the threshold value is equal to said second pressure difference value between the second pressure value P₂ inside the transmission system 20 and the third pressure measured outside the transmission system.

In addition, the present invention applies to a utilization of the device according to the invention or support according to the invention previously connected to the control module, on which the injector is previously loaded, for performing a surgical operation to implant an intraocular lens through a corneal incision comprising the following steps:
a first pre-folding step, in which said control module 10 is activated so that the actuator 30 is displaced from said first rest position to a second lens pre-folding position corresponding to a first state in which the lens is housed in pre-folded form in a rolling area defined in one part of the inner section of said cartridge; and
a second injection step, in which said control module is activated so that the actuator is displaced from said second lens pre-folding position to a third injection position, said second step corresponding to a second state in which said lens is driven outside of said cartridge.

Advantageously, prior to the step in which control module 10 is activated, the utilization of the device or support connected to control module 10 also involves a third additional step in which the cartridge is assembled on the injector connection area.

The utilization of the device or support is also characterized in that, during the second injection step, control module 10 can be activated so that actuator means 60 is displaced from the third injection position to the second lens pre-folding position.

In the context of the utilization of the device according to the invention to perform a surgical operation, the practitioner should follow the following assembly procedure before proceeding to said operation:
assembly of cartridge A (previously loaded or pre-loaded with the lens) on connection head 81 of injector 50.
assembly of injector 50 on the support of said sub-assembly B;
and optionally, connection (for example by using the sterile device) of the first sealed part 201 of sub-assembly B to the second sealed part 203 of sub-assembly C.

It is understood that the present invention is in no way limited to the embodiments described above and that modifications may be made without departing from the scope of the appended claims.

1-29. (canceled)

30. A control device for controlling an intraocular lens injector, said control device comprising:
a hydraulic pedal, and
a hermetic incompressible fluid transmission system for connecting said hydraulic pedal to said injector, said hydraulic pedal being arranged to activate an injector by applying pressure to said transmission system fluid so as to generate a pressure force intended to operate the injector,
said control device being characterized in that it comprises a support arranged to accommodate the injector, said support comprising an actuator arranged to be in contact with and to cooperate with the injector, said actuator being connected to the fluid transmission system and, as a result of said fluid pressure force, being arranged to be moved between a first rest position and at least one second position.

31. A control device according to claim 30, characterized in that said incompressible fluid is a liquid.

32. A control device according to claim 31, characterized in that said actuator comprises a contact surface arranged to enter into contact with a surface of the injector, said contact surface being defined by a predetermined contact area value, and in that, for a predetermined pressure value constantly applied to said fluid through said hydraulic pedal, said pressure force of said fluid is proportional to said area value of said contact surface.

33. A control device according to claim 30, characterized in that said actuator comprises a contact surface arranged to enter into contact with a surface of the injector, said contact surface being defined by a predetermined contact area value, and in that, for a predetermined pressure value constantly applied to said fluid through said hydraulic pedal, said pressure force of said fluid is proportional to said area value of said contact surface.

34. A control device according to claim 30, characterized in that said fluid transmission system comprises at least one pipe communicating, at a first end, with said hydraulic pedal and, at a second end, with said actuator of said support, such that said fluid may circulate between said hydraulic pedal and said actuator.

35. A control device according to claim 31, characterized in that said fluid transmission system comprises at least one pipe communicating, at a first end, with said hydraulic pedal and, at a second end, with said actuator of said support, such that said fluid may circulate between said hydraulic pedal and said actuator.

36. A control device according to claim 30, characterized in that said fluid transmission system comprises a first sealed part connected to the actuator of said support and arranged to be connected by a connection means to at least one second sealed part of said fluid transmission system, said connection means being arranged to unseal each part of the transmission system to form a fluid transmission system which is hermetic and through which the fluid from each part of the fluid transmission system is arranged to circulate, said second sealed part of said transmission system being connected to a hydraulic pedal.

37. A control device according to claim 31, characterized in that said fluid transmission system comprises a first sealed part connected to the actuator of said support and arranged to be connected by a connection means to at least one second sealed part of said fluid transmission system, said connection means being arranged to unseal each part of the transmission system to form a fluid transmission system which is hermetic and through which the fluid from each part of the fluid transmission system is arranged to circulate, said second sealed part of said transmission system being connected to a hydraulic pedal.

38. A control device according to claim 30, characterized in that said actuator comprises a piston assembled in a cylinder comprising a first end communicating with said fluid transmission system, said piston being lengthened by an arm which extends outside of said cylinder through an opening provided on a second cylinder end, opposite to the first cylinder end, said arm comprising an end arranged to enter in contact and cooperate with a part of an injector.

39. A control device according to claim 31, characterized in that said actuator comprises a piston assembled in a cylinder comprising a first end communicating with said fluid transmission system, said piston being lengthened by an arm which extends outside of said cylinder through an opening provided on a second cylinder end, opposite to the first cylinder end, said arm comprising an end arranged to enter in contact and cooperate with a part of an injector.

40. A control device according to claim 30, characterized in that an anti-bubble module comprising a reservoir, arranged to contain a first pressurized vector fluid part, is connected by a second transmission system to said fluid transmission system through a second connection means, so that the first vector fluid part can circulate between the reservoir and the transmission system comprising a second vector fluid part, the anti-bubble module also comprising a non-return valve arranged to control the direction of circulation of the vector fluid by enabling the passage of this fluid in a first direction defined from the reservoir to said fluid transmission system, while blocking the circulation of said vector fluid in a direction reverse to said first direction.

41. A control device according to claim 31, characterized in that an anti-bubble module comprising a reservoir, arranged to contain a first pressurized vector fluid part, is connected by a second transmission system to said fluid transmission system through a second connection means, so that the first vector fluid part can circulate between the reservoir and the transmission system comprising a second vector fluid part, the anti-bubble module also comprising a non-return valve arranged to control the direction of circulation of the vector fluid by enabling the passage of this fluid in a first direction defined from the reservoir to said fluid transmission system, while blocking the circulation of said vector fluid in a direction reverse to said first direction.

42. A control device according to claim 30 characterized in that the support comprises an indentation arranged to accommodate an injector arranged to be detachably housed therein.

43. An assembly comprising:
a control device according to claim 30, and
an intraocular lens injector.

44. A method for operating a control device according to claim 30, comprising:
a step in which said hydraulic pedal is activated so as to apply pressure on said fluid;
a step in which, as a result of the pressure applied by said hydraulic pedal, a pressure force is generated, this pressure force being intended to operate the injector,
characterized in that said pressure force is exerted on the injector through the actuator of said support, said actuator, as a result of said fluid pressure force, being arranged to be moved between a first rest position and at least one second position.

45. A method according to claim 44, comprising a step in which a cartridge, in which an intraocular lens is pre-loaded, is assembled on a connection area of the injector.

46. A method according to claim 45, characterized by the following successive steps:
a first pre-folding step, in which said hydraulic pedal is activated so that the actuator is displaced from said first rest position to a second lens rolling position corresponding to a first state in which the lens is housed in a rolling area defined in one part of the inner section of said cartridge in pre-folded form; and
a second injection step, in which said hydraulic pedal is activated so that the actuator is displaced from said second lens rolling position to a third lens injection position, said second step corresponding to a second state in which said lens is driven outside of said cartridge.

47. A method according to claim 46, characterized in that the operating method is also characterized in that, during the second injection step, said hydraulic pedal is activated so that said actuator is displaced from said third injection position to said second pre-folding position.

48. A method according to claim 44 for operating a control device according to claim 40, characterized in that the method also comprises a step in which, when a difference value ΔP between a first pressure value (P₁) of a first vector fluid part contained in the reservoir of the anti-bubble module and a second pressure value (P₂) of a second vector fluid part contained in the transmission system of the control device is at least equal to a threshold value, the vector fluid contained in the reservoir circulates in a first direction, from said reservoir to said transmission system, so that the difference value ΔP is less than said threshold value.

49. A method according to claim 48, characterized in that said pressure difference value ΔP=P₁−P₂ is at least equal to a second pressure difference value between the second pressure value (P₂) inside the fluid transmission system and a third pressure measured outside the transmission system.
